(19) United States
(12) Patent Application Publication
(10) Pub. No.: US 2025/0170286 A1
(43) Pub. Date: May 29, 2025

(54) SANITIZING AND DEODORIZING DEVICE

(71) Applicant: HCL Technologies Limited, New Delhi (IN)

(72) Inventors: ROHIT SUREKH, Bangalore (IN); ACHAL P M KAILASH, Bangalore (IN); CHARANDEEP KAPOOR, Bangalore (IN)

(21) Appl. No.: 18/631,139

(22) Filed: Apr. 10, 2024

(30) Foreign Application Priority Data
Nov. 28, 2023 (IN) 202311080678

(51) Int. Cl. A61L 2/06; A61L 2/26; A61L 9/014

(52) U.S. Cl. CPC A61L 2/06; A61L 2/26; A61L 9/014; A61L 2202/15; A61L 2202/26; A61L 2209/22

(57) ABSTRACT

This disclosure relates to a sanitizing and deodorizing device. The device may include a wireless power source detachably connected to the device for powering the device. The device may further include a control knob configured to facilitate a user to switch between a shoe sanitizing mode and a bag deodorizing mode. The device may further include an activated charcoal configured to absorb odor and moisture from a shoe in the shoe sanitizing mode, and odor from a bag in the bag deodorizing mode. The device may further include a heating coil, selectively activated in the shoe sanitizing mode, configured to heat dry air from the activated charcoal to a user-defined temperature. The device may further include an exhaust fan positioned in between the activated charcoal and the heating coil configured to maintain a continuous airflow throughout one of the shoe sanitization and bag deodorization.

### TECHNICAL FIELD

This disclosure relates generally to hygiene and personal care devices, and more specifically to a device designed for sanitizing shoes and deodorizing bags.

### Background

As per data, an average foot may sweat up to a cup daily. Extensive sweating may lead to fungal infections. The shoe may transfer up to 90% to 99% bacteria to home. An average of 2887 units of bacteria reside inside the shoes. The deodoux helps individuals to cultivate the habit of foot hygiene by using sanitized shoes each time. Deodoux can be extensively helpful for hyperhidrosis patients (Hyperhidrosis—is excessive sweating that's not always related to heat or exercise, which is common in diabetic patients). In today's fast-paced world, individuals often find themselves facing challenges in maintaining proper hygiene, especially when it comes to their footwear and bags. Excessive sweating, exposure to bacteria, and the accumulation of moisture may lead to foul odors, bacterial infections, and the transfer of pathogens into living spaces. Conventional device of shoe and bag maintenance typically require dedicated time and physical effort, which may be impractical for busy individuals.

According to available data, an average person's feet may produce up to a cup of sweat daily, leading to potential fungal infections. Furthermore, shoes have been identified as significant carriers of bacteria, with estimates suggesting that they may transfer up to 90% to 99% of the bacteria encountered throughout the day into one's home. Inside the average shoe, a staggering 2887 units of bacteria may reside, posing a considerable risk to foot health and overall hygiene.

Therefore, in order to provide solutions to the aforementioned drawback, there exists a need to develop a sanitizing and deodorizing device that revolutionizes personal footwear and bag hygiene. The proposed device not only promotes personal hygiene but also addresses the needs of individuals with hyperhidrosis (i.e., excessive sweating) and others seeking a hassle-free solution for maintaining clean and odor-free shoes and bags.

### SUMMARY

In one embodiment, a sanitizing and deodorizing device is disclosed. The device may include a wireless power source detachably connected to the device for powering the device. The device may further include a control knob configured to facilitate a user to switch between a shoe sanitizing mode and a bag deodorizing mode. The device may further include an activated charcoal configured to absorb odor and moisture from a shoe in the shoe sanitizing mode, and odor from a bag in the bag deodorizing mode. The device may further include a heating coil, selectively activated in the shoe sanitizing mode, configured to heat dry air from the activated charcoal to a user-defined temperature. The device may further include an exhaust fan positioned in between the activated charcoal and the heating coil configured to maintain a continuous airflow throughout one of the shoe sanitization and bag deodorization.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a perspective view of a sanitizing and deodorizing device, in accordance with some embodiments of the present disclosure.

FIG. 2 illustrates an exploded view of a sanitizing and deodorizing device, in accordance with some embodiments of the present disclosure.

FIG. 3A illustrates a perspective view of a sanitizing and deodorizing device operating in a shoe sanitizing mode, in accordance with an exemplary embodiment of the present disclosure.

FIG. 3B illustrates a perspective view of a sanitizing and deodorizing device operating in a bag deodorizing mode, in accordance with an exemplary embodiment of the present disclosure.

FIG. 4A illustrates a placement of sanitizing and deodorizing device inside a shoe to operate in a shoe sanitizing mode, in accordance with an exemplary embodiment of the present disclosure.

FIG. 4B illustrates a placement of sanitizing and deodorizing device inside a bag to operate in a bag deodorizing mode, in accordance with an exemplary embodiment of the present disclosure.

FIG. 5 illustrates a magnetic pairing mechanism for pairing at least two sanitizing and deodorizing devices, in accordance with an exemplary embodiment of the present disclosure.

FIG. 6A illustrates removal of an air outlet vent from a sanitizing and deodorizing device for cleaning purposes, in accordance with some embodiments of the present disclosure.

FIG. 6B illustrates a sanitizing and deodorizing device with replacement of an activated charcoal, in accordance with some embodiments of the present disclosure.

### DETAILED DESCRIPTION

The following description is presented to enable a person of ordinary skill in the art to make and use the invention and is provided in the context of particular applications and their requirements. Various modifications to the embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention might be practiced without the use of these specific details. In other instances, well-known structures and devices are shown in block diagram form in order not to obscure the description of the invention with unnecessary detail. Thus, the invention is not intended to be limited to the embodiments shown but is to be accorded the widest scope consistent with the principles and features disclosed herein.

While the invention is described in terms of particular examples and illustrative figures, those of ordinary skill in the art will recognize that the invention is not limited to the examples or figures described. Those skilled in the art will recognize that the operations of the various embodiments may be implemented using hardware, software, firmware, or combinations thereof, as appropriate. For example, some processes can be carried out using processors or other digital circuitry under the control of software, firmware, or hard-wired logic. (The term “logic” herein refers to fixed hardware, programmable logic and/or an appropriate combination thereof, as would be recognized by one skilled in the art to carry out the recited functions.) Software and firmware can be stored on computer-readable storage media. Some other processes can be implemented using analog circuitry, as is well known to be one of ordinary skill in the art. Additionally, memory or other storage, as well as communication components, may be employed in embodiments of the invention.

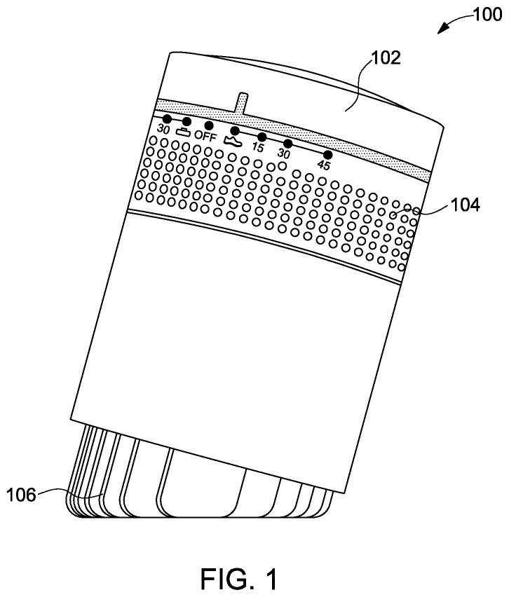

Referring now to FIG. 1, a perspective view of a sanitizing and deodorizing device 100 is illustrated, in accordance with some embodiments of the present disclosure. The present FIG. 1 depicts a control knob 102 configured to switch between a shoe sanitizing mode and a bag deodorizing mode, an air inlet vent 104 configured to intake fresh air from atmosphere, and an air outlet vent 106 designed with a slant to allow maximum airflow to a toe end of the shoe during shoe sanitizing mode. Apart from switching between the shoe sanitizing mode and the bag deodorizing mode, the control knob 102 may also be used to turn on and off the device 100.

The sanitizing and deodorizing device 100 revolutionizes personal footwear and bag hygiene. The sanitizing and deodorizing device 100 may operate in two distinct modes i.e., in the shoe sanitizing mode and the bag deodorizing mode.

In the shoe sanitizing mode, the sanitizing and deodorizing device 100 may employ a combination of an exhaust fan, an activated charcoal (not shown in FIG. 1) and a heating coil (not shown in FIG. 1) to remove moisture, eliminate bacteria, and deodorize shoes, all without the use of chemicals. The unique slanted vent design of the air outlet vent 106 ensures maximum airflow to the toe end of the shoe, enhancing the overall sanitation process and extending the life of shoes.

In bag deodorizing mode, the sanitizing and deodorizing device 100 may run the exhaust fan to circulate fresh air within a bag through activated charcoal, effectively deodorizing the bag and enhancing their overall freshness. The sanitizing and deodorizing device 100 may be conveniently powered by a wireless power bank, ensuring portability and usability on the go.

A user-friendly control knob 102 allows for mode selection and timer customization, offering flexibility and ease of use. With its chemical-free approach, adjustable features, and efficient design, the proposed sanitizing and deodorizing device 100 represents a significant advancement in the field of personal hygiene and care devices, offering a convenient and effective solution for individuals in a fast-moving world.

The sanitizing and deodorizing device 100 is designed with versatility in mind when it comes to its power source. In some embodiments, the device 100 may be powered by the wireless power bank, providing users with the convenience of a portable and rechargeable energy supply. This wireless power bank ensures that the device 100 may be easily moved and used without the constraints of a wired connection.

Alternatively, in certain embodiments, the sanitizing and deodorizing device 100 may also have the capability to be powered directly through a a wired power source (e.g., USB cable). This option offers users flexibility in selecting their power source, allowing them to connect the device 100 to a standard USB port, ensuring continuous operation without the need for the wireless power bank. These power source options enhance the device adaptability and usability, catering to user preferences and situations, whether they prefer the convenience of a wireless power bank, or the direct connectivity offered by a USB cable.

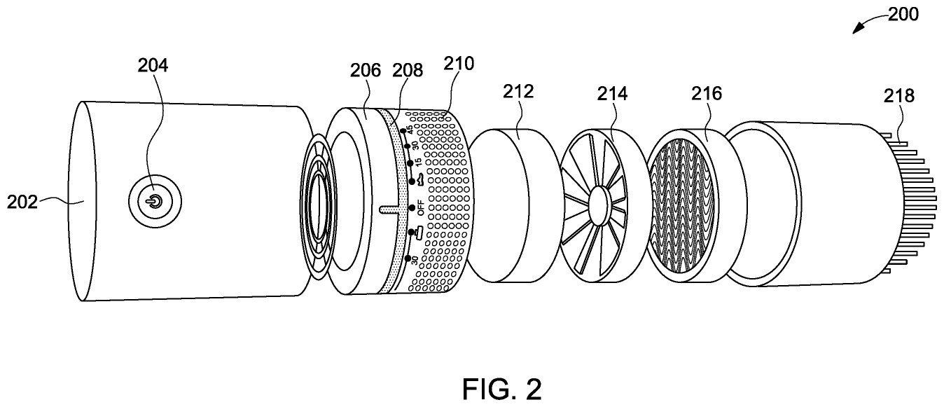

Referring now to FIG. 2, an exploded view of a sanitizing and deodorizing device 200 is illustrated, in accordance with some embodiments of the present disclosure. The present FIG. 2 provides a detailed view of various components that make up the sanitizing and deodorizing device 200. These components work in coordination to deliver an efficient and user-friendly solution for sanitizing and deodorizing both shoes and bags, promoting personal hygiene and enhancing the lifespan of these everyday items.

The sanitizing and deodorizing device 200 may include a wireless power source (e.g., a magnetic wireless power bank 202), a power bank switch and battery level indicator 204, a control knob 206, a mode indicator light 208, an air inlet vent 210, an activated charcoal 212, an exhaust fan 214, a heating coil 216, and an air outlet vent 218.

At the core of the device 200 is the magnetic wireless power bank 202 (e.g., dual sided power bank) that serves as the power source. This power bank is designed to be detachable, allowing for easy replacement and recharging as needed. It provides the necessary electrical power to operate the device 200 during both shoe sanitization and bag deodorization processes.

The power bank switch and battery level indicator 204 located on the wireless power bank 202 may provide users with real-time information about remaining battery life of the wireless power bank 202. This feature ensures that users are aware of the device power status and may plan for recharging accordingly.

The control knob 206 is a user interface element that plays an important role in operating the sanitizing and deodorizing device 200. Users may interact with the control knob 206 to select a desired mode, whether it be a shoe sanitizing mode or a bag deodorizing mode. Additionally, the control knob 206 may allow users to adjust predefined timers for sanitization and deodorization, adapting the device operation to their specific needs.

By way of an example, users may adjust predefined timers for sanitization and deodorization with a simple turn of the control knob 206. Suppose a user desires a shorter sanitization cycle for their shoes, perhaps in situations where they need a quick refresh before heading out. In such a scenario, the user rotates the control knob 206 to select a shorter timer duration, such as 15 seconds.

Conversely, for instances where a more thorough sanitization or deodorization process is required, such as after a long day or in the case of particularly odorous shoes or bags, users may rotate the control knob 206 to choose a longer timer duration, such as 30 seconds. This extended duration allows the device 200 to ensure that moisture is effectively removed, bacteria are eliminated, and odors are neutralized.

The control knob flexibility in adjusting timer settings empowers users to adapt the sanitizing and deodorizing process to their immediate needs, striking a balance between speed and thoroughness. Whether it is a quick refresh before a meeting or a more extensive treatment after a workout, the control knob 206 puts control in the hands of the user, enhancing the device versatility and adaptability.

The mode indicator light 208 on the control knob 206 serves as a visual indicator to inform users of the current operating mode of the sanitizing and deodorizing device 200. A specific color may be associated with each mode, with one color (e.g., yellow color) indicating shoe sanitizing mode and another color (e.g., blue color) indicating bag deodorizing mode, providing users with clear feedback on the device status.

The air inlet vent 210 is designed to intake fresh air from the surrounding environment. This fresh air plays a crucial role in the device operation, facilitating the circulation of air through the activated charcoal 212 and ensuring effective sanitization and deodorization.

The activated charcoal 212 is a key component of the sanitizing and deodorizing device 200, responsible for absorbing moisture and odor from shoes and bags. It serves as a natural and chemical-free deodorizing agent, enhancing the freshness of the items undergoing treatment.

The heating coil 216 may be selectively activated when the sanitizing and deodorizing device 200 is in the shoe sanitizing mode. It plays a vital role in elevating the temperature of the dry air from the activated charcoal to approximately 45 to 65 degrees Celsius. This controlled heat not only removes moisture but also helps in eradicating bacteria, ensuring thorough shoe sanitization.

The exhaust fan 214 may be strategically placed in between the activated charcoal 212 and the heating coil 216 within the sanitizing and deodorizing device 200 to maintain a continuous and controlled airflow throughout the sanitization and deodorization processes. This constant airflow is essential for the effective functioning of the sanitizing and deodorizing device 200.

The air outlet vent 218 may be a removable vent designed with a slanted configuration. This unique design promotes maximum airflow to the toe end of shoes during sanitization, ensuring that all areas of the shoe receive proper treatment. The air outlet vent 218 may be easily removed and cleaned for maintenance.

In this way, the sanitizing and deodorizing device 200 provides a wide range of scenarios and preferences, ensuring that users may consistently enjoy the benefits of clean, fresh shoes and bags, with each time cycle adapted to their specific needs.

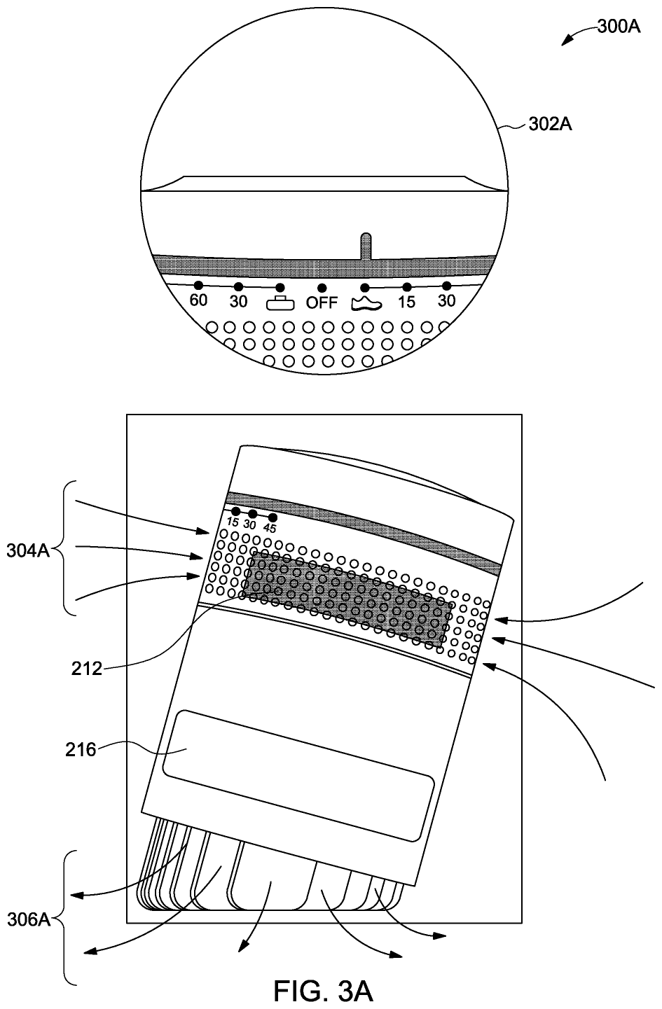

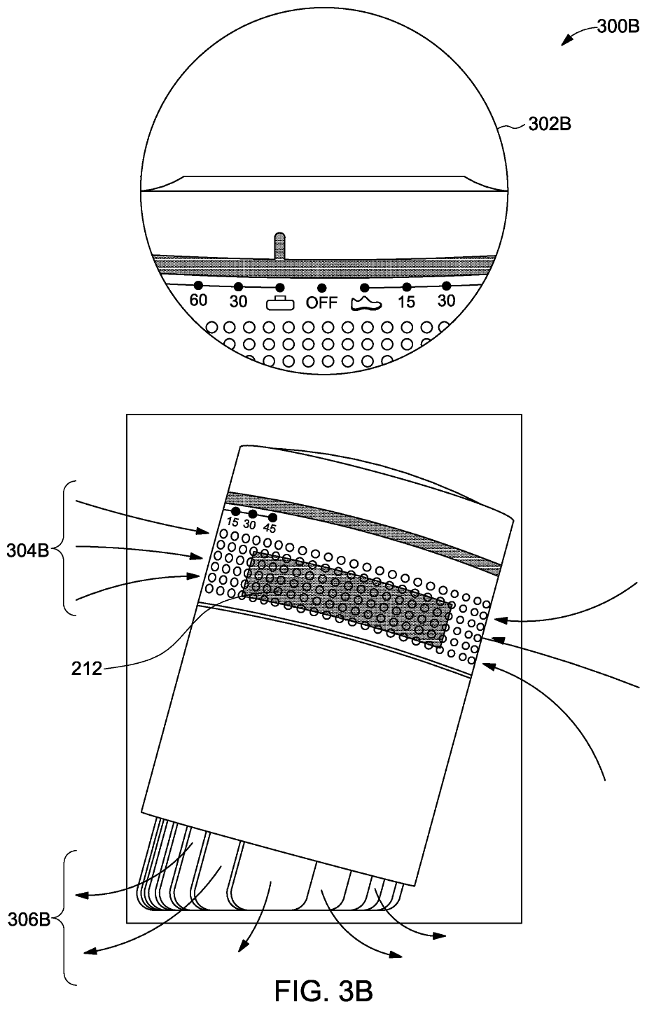

Referring now to FIG. 3A, a perspective view of a sanitizing and deodorizing device 300A operating in a shoe sanitizing mode 302A is illustrated, in accordance with an exemplary embodiment of the present disclosure. The shoe sanitizing mode 302A is designed to thoroughly sanitize shoes, ensuring they are clean, fresh, and free from moisture and bacteria.

To initiate the shoe sanitizing mode 302A, initially the sanitizing and deodorizing device 300A may be placed inside a shoe that requires sanitization. This step ensures that the device 300A is properly positioned to treat the interior of the shoe effectively.

Further, the control knob 206 may be rotated to select the shoe sanitizing mode 302A. This action activates the device 300A and configures it for shoe sanitization, setting the stage for the sanitization process to commence with an operation indicator light (e.g., yellow light) signalling the mode's activation.

While operating in the shoe sanitizing mode 302A, the air inlet vent 210, positioned within the device 300A, comes into play. It serves as the gateway for fresh air 304A from the surrounding environment to enter the device 300A. This fresh air intake is a crucial part of the process, as it ensures that the shoe is exposed to clean and uncontaminated air.

The incoming fresh air 304A then proceeds to pass through the activated charcoal 212 within the device 300A. The activated charcoal 212 that initially absorbed the odor from the shoe may further effectively de-moisturize the incoming air, removing any excess moisture or humidity present in the shoe. This step is essential for addressing one of the key issues in footwear hygiene-moisture buildup, which may lead to bacterial growth.

After passing through the activated charcoal 212, the de-moisturized air may now enter the heating coil 216. In this component of the device 300A, the air may be heated to a temperature that is user-defined, preferably within the range of 45-65 degrees Celsius.

This controlled heating may serve multiple purposes, for example, a warm temperature effectively kills bacteria and pathogens that may be present within the shoe. Moreover, any remaining moisture in the shoe may further removed during this step.

Following the heating process, the de-moisturized warm air 306A, now sanitized and refreshed, may be released from the device 300A through the air outlet vent 218. This vent is uniquely designed to promote maximum airflow to the toe end of the shoe, ensuring that all areas of the shoe receive thorough treatment.

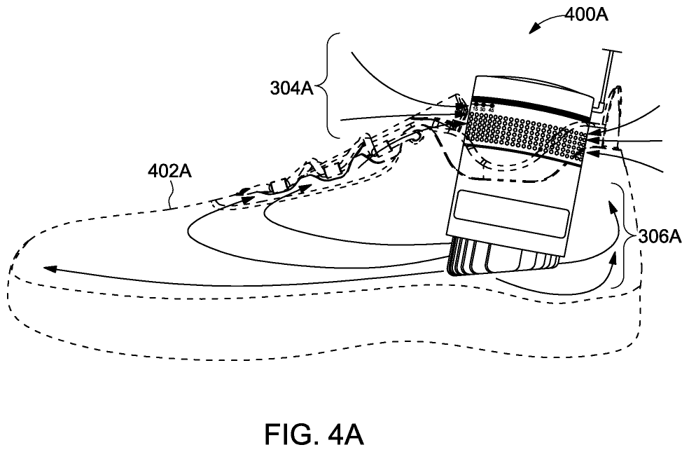

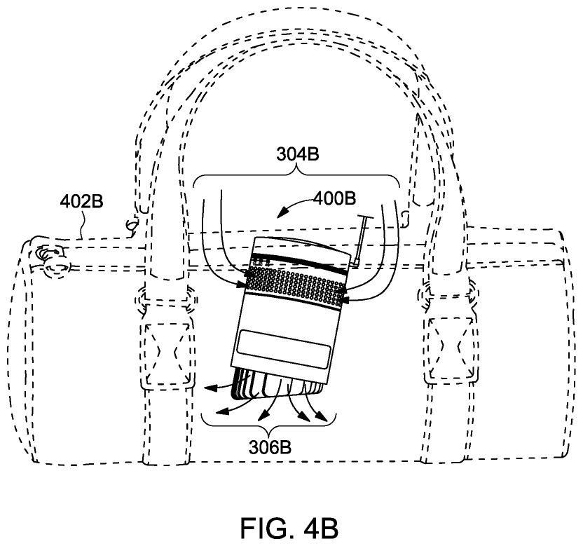

By way of an example, as depicted in FIG. 4A, a sanitizing and deodorizing device 400A placed inside a shoe 402A is operating in a shoe sanitizing mode. The shoe sanitizing mode of the sanitizing and deodorizing device 400A is designed to sanitize and remove moisture from the shoe 402A. It utilizes fresh air intake 304A, activated charcoal de-moisturization, controlled heating, and precise airflow 306A dispersion to ensure that the shoe 402A is left in optimal condition, promoting both hygiene and comfort.

Referring now to FIG. 3B, a perspective view of a sanitizing and deodorizing device 300B operating in a bag deodorizing mode 302B, in accordance with an exemplary embodiment of the present disclosure. The bag deodorizing mode 302B is specifically designed to address the hygiene and freshness of a bag, ensuring the bag is free from unpleasant odors. The present FIG. 3B provides an overview of how the device 300B may function during bag deodorizing mode 302B.

The sanitizing and deodorizing device 300B may be configured to deodorize the bag. It may be placed inside the bag that requires deodorization, positioned strategically to treat the interior of the bag effectively.

The control knob 206 may be rotated to select the deodorizing mode 302B. This action activates the device 300B and configures it for bag deodorization, with an operation indicator light (e.g., blue light) signalling the mode's activation, ensuring users are aware of the device's current operation.

While operating in the bag deodorizing mode 302B, the device 300B may draw in fresh air 304B from the surrounding environment. This fresh air 304B may enter the device 300B through an air inlet vent 210, which facilitates the circulation of air through an activated charcoal 212 within the device 300B.

The activated charcoal 212 may serve as a crucial component during bag deodorization. It may effectively absorb any odors present within the bag, leaving it smelling clean and fresh.

After passing through the activated charcoal 212, the deodorized air 306B exits the device 300B through an air outlet vent 218. This vent is designed to ensure that the deodorized air 306B is evenly distributed throughout the interior of the bag, effectively addressing odors and enhancing overall bag freshness.

By way of an example, as depicted in FIG. 4B, a sanitizing and deodorizing device 400B is placed inside a bag 402B operating in bag deodorizing mode. This mode is designed to address the specific needs of the bag 402B, effectively deodorizing them and ensuring that they remain free from unpleasant odors. The device operation in this mode is characterized by the intake of fresh air 304B, the use of activated charcoal, and the even distribution of deodorized air 306B within the bag, all contributing to enhanced bag hygiene and user comfort. It should be noted that in bag deodorizing mode, heating coil may not be operational, considering a long duration of working time on power bank.

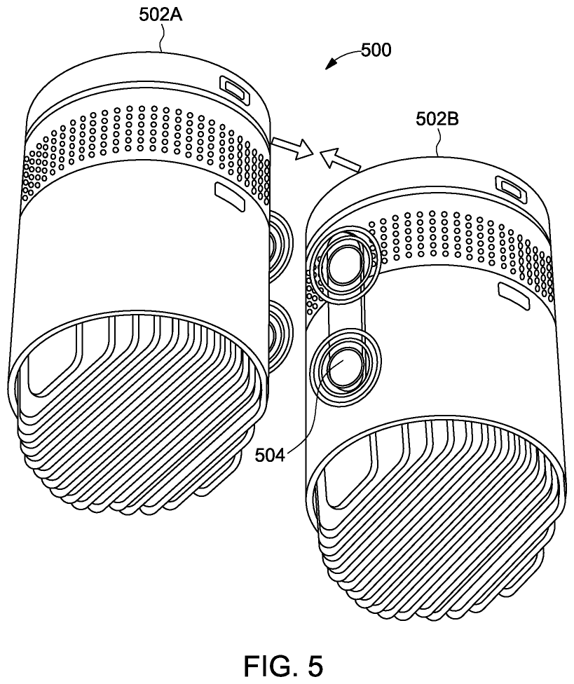

Referring now to FIG. 5, a magnetic pairing mechanism 500 for pairing at least two sanitizing and deodorizing devices, in accordance with an exemplary embodiment of the present disclosure. This mechanism is a unique feature that enhances the convenience and organization of multiple devices.

As depicted in the present FIG. 5, the two sanitizing and deodorizing devices (for example, 502A and 502B) are equipped with magnetic pairing capability. Each device is strategically designed to be paired with another, simplifying the process of organizing and storing multiple devices together.

The magnetic pairing mechanism 500 is characterized by magnetic elements 504 incorporated into the devices 502A and 502B, often located on the exterior or sides of the devices 502A and 502B. When two devices 502A and 502B are brought into close proximity, these magnetic elements 504 create a secure and stable connection between them, effectively pairing the devices.

By pairing two sanitizing and deodorizing devices 502A and 502B using a magnetic connection, users may conveniently organize and store the devices when not in use. This feature ensures that the devices 502A and 502B are readily available and well-organized for future sanitization and deodorization tasks.

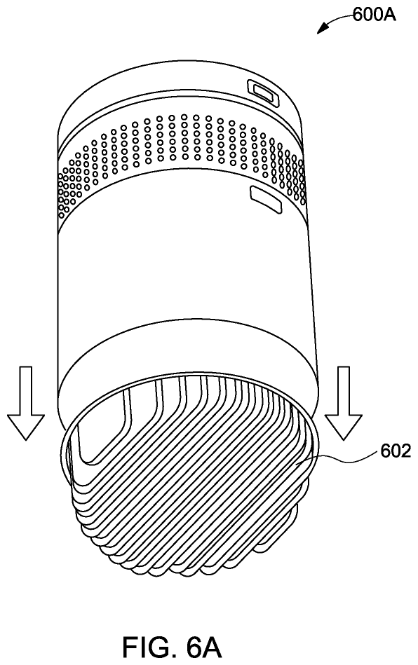

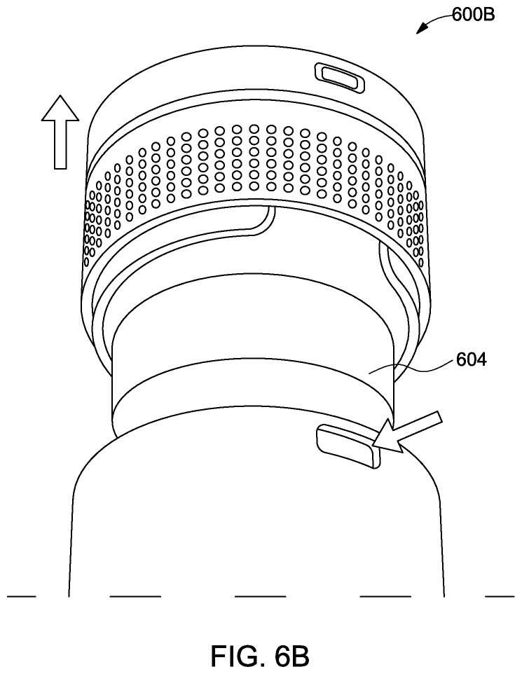

Referring now to FIG. 6A, a sanitizing and deodorizing device 600A with removal of an air outlet vent 602 for cleaning purposes is illustrated, in accordance with some embodiments of the present disclosure. This feature may serve a practical purpose in the maintenance and upkeep of the device 600A. The practical feature of being able to remove the air outlet vent 602 for cleaning purposes, enhancing the user experience, and ensuring that the device 600A remains efficient and reliable throughout its lifespan.

The sanitizing and deodorizing device 600A is depicted with an emphasis on the air outlet vent 602, which is designed with a removable feature for easy cleaning and maintenance. In particular, the present FIG. 6A demonstrates how the air outlet vent 602 may be detached from the device 600A, providing users with convenient access to this component.

The ability to remove the air outlet vent 602 simplifies the process of cleaning and maintaining the device 600A. Users may easily clean the vent to ensure that it remains free from dust, debris, or any accumulated particles that might hinder airflow.

Moreover, regular maintenance, including the cleaning of components like the air outlet vent 602, contributes to the longevity and optimal performance of the sanitizing and deodorizing device 600A.

Referring now to FIG. 6B, a sanitizing and deodorizing device 600B with replacement of an activated charcoal 604 is illustrated, in accordance with some embodiments of the present disclosure. This feature serves a practical purpose in maintaining the effectiveness of the device 600B. The practical feature of being able to replace the activated charcoal 604, contributing to the device longevity and its ongoing ability to effectively eliminate odors and absorb moisture from shoes and bags, ultimately enhancing user satisfaction.

The sanitizing and deodorizing device 600B is shown with a focus on the activated charcoal 604, which is designed to be replaceable. In particular, the present FIG. 6B demonstrates how the activated charcoal 604 may be taken out and replaced with fresh activated charcoal when needed.

Over time, activated charcoal 604 may become saturated with moisture or odors from shoes and bags. By offering the capability to replace the activated charcoal 604, the device 600B ensures that it may consistently provide effective de-moisturization and deodorization, promoting shoe and bag hygiene.

The ability to replace the activated charcoal 604 allows users to extend the life of the device 600B, as they may refresh its key component rather than replacing the entire device, offering a cost-effective solution.

It should be noted that the sanitizing and deodorizing device illustrated in various embodiments of the present disclosure may be analogous. For example, the sanitizing and deodorizing device 100 of FIG. 1 may share analogous features and functionalities with the sanitizing and deodorizing devices 200, 300A, 300B, 400A, 400B, 502A, 502B, 600A, and 600B presented in other figures of this disclosure. These analogous devices, while possibly varying in specific design and implementation details, share the common goal of providing effective sanitization and deodorization solutions for shoes and bags, to meet diverse user needs and preferences.

As will be appreciated by those skilled in the art, the sanitizing and deodorizing device described in the various embodiments discussed above are not routine, or conventional, or well understood in the art. The device discussed above provide for sanitizing and deodorizing shoes and bags. The device discussed above provide various advantages, such as enhanced efficiency, user-friendliness, versatility, simultaneous treatment, cost-effective maintenance, and optimized longevity. The proposed device contributes to improved hygiene, comfort, and user satisfaction.

The combination of activated charcoal, heating coil, and exhaust fan in the sanitizing and deodorizing device ensures efficient de-moisturization and deodorization. It eliminates excess moisture from shoes, preventing bacterial growth and unpleasant odors, while also effectively deodorizing bags, leaving them fresh and odor-free.

Further, the inclusion of a control knob with predefined timers simplifies the operation of the sanitizing and deodorizing device, making it user-friendly and accessible to individuals of all levels of technical expertise.

Further, with dual modes for shoes and bags, the device offers a wide range of user needs, providing a versatile solution for maintaining hygiene and freshness in footwear and bags.

Further, the sanitizing and deodorizing device allows for the simultaneous sanitization of a pair of shoes, which is especially advantageous when time is of the essence, ensuring that both shoes are thoroughly cleaned and deodorized together.

Further, the capability to replace activated charcoal ensures that the device remains cost-effective in the long run. Instead of replacing the entire device, users may simply refresh the activated charcoal, reducing operational costs.

Furthermore, by enabling users to remove and clean the air outlet vent, the sanitizing and deodorizing device enhances the longevity by facilitating regular maintenance, ensuring consistent and efficient airflow.

In light of the above-mentioned advantages and the technical advancements provided by the disclosed device, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

Although the present invention has been described in connection with some embodiments, it is not intended to be limited to the specific form set forth herein. Rather, the scope of the present invention is limited only by the claims. Additionally, although a feature may appear to be described in connection with particular embodiments, one skilled in the art would recognize that various features of the described embodiments may be combined in accordance with the invention.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

It is intended that the disclosure and examples be considered as exemplary only, with a true scope and spirit of disclosed embodiments being indicated by the following claims.

What is claimed is:

1. A sanitizing and deodorizing device, the device comprising:
a wireless power source detachably connected to the device for powering the device;
a control knob configured to facilitate a user to switch between a shoe sanitizing mode and a bag deodorizing mode;
an activated charcoal configured to absorb odor and moisture from a shoe in the shoe sanitizing mode, and odor from a bag in the bag deodorizing mode;
a heating coil, selectively activated in the shoe sanitizing mode, configured to heat dry air from the activated charcoal to a user-defined temperature; and
an exhaust fan positioned in between the activated charcoal and the heating coil configured to maintain a continuous airflow throughout one of the shoe sanitization and bag deodorization.

2. The device of claim 1, further comprising:
an air inlet vent configured to intake fresh air from atmosphere; and
an air outlet vent designed with a slant to allow maximum airflow to a toe end of the shoe during shoe sanitizing mode.

3. The device of claim 1, wherein the control knob is further configured to enable the user to select a set of predefined timers for sanitization and deodorization.

4. The device of claim 1, wherein in the shoe sanitizing mode, dry air from the activated charcoal passes through the heating coil reaches the user-defined temperature to remove moisture, kill bacteria, and sanitize the shoe.

5. The device of claim 1, wherein in the bag deodorizing mode, the exhaust fan circulates fresh air in the bag via the activated charcoal to deodorize the bag when connected to the wireless power source.

6. The device of claim 1, wherein the heating coil is deactivated in bag deodorizing mode.

7. The device of claim 1, wherein the wireless power source detachably connected to the device, facilitates easy replacement, and recharging of the power source.

8. The device of claim 1, further comprising an indicator light to indicate operational modes of the device, wherein a first colour indicates an activation of the shoe sanitizing mode, and a second colour indicates an activation of the bag deodorizing mode.

9. The device of claim 1, wherein the activated charcoal is replaceable to ensure optimal performance.

10. The device of claim 1, wherein the air outlet vent is removable for easy cleaning and maintenance.

11. The device of claim 1, further comprising a magnetic pairing mechanism, wherein the magnetic pairing mechanism facilitates at least two sanitizing and deodorizing devices to be wirelessly paired together for simultaneous sanitizing of a pair of shoes.
